(19) United States
(12) Patent Application Publication
(10) Pub. No.: US 2023/0241028 A1
(43) Pub. Date: Aug. 3, 2023

(54) TREATMENT OF OVARIAN CANCER WITH NIROGACESTAT

(71) Applicant: SpringWorks Therapeutics, Inc., Stamford, CT (US)

(72) Inventors: Shinta CHENG, Stamford, CT (US); Todd SHEARER, Stamford, CT (US)

(21) Appl. No.: 18/162,821

(22) Filed: Feb. 1, 2023

Related U.S. Application Data
(60) Provisional application No. 63/267,413, filed on Feb. 1, 2022.

(51) Int. Cl. A61K 31/4164; A61K 9/00; A61K 9/20; A61P 35/00

(52) U.S. Cl. CPC A61K 31/4164; A61K 9/20; A61K 9/0053; A61P 35/00

(57) ABSTRACT

The present disclosure relates to methods for treating ovarian cancer comprising nirogacestat or a pharmaceutically acceptable salt thereof.

### CROSS REFERENCE TO RELATED APPLICATIONS

The present application claims the benefit of priority to U.S. Provisional Application No. 63/267,413, filed on Feb. 1, 2022, the contents of which are hereby incorporated by reference in its entirety.

### FIELD OF THE INVENTION

The present disclosure relates to methods for treating ovarian cancer comprising administering to a patient in need thereof nirogacestat or a pharmaceutically acceptable salt thereof.

### BACKGROUND

Ovarian granulosa cell tumors (OvGCTs) represent 5-7% of all ovarian cancers (approximately 1500 to 2000 newly diagnosed patients/year in the United States (US)) and are the most common subtype of ovarian sex cord tumors (70%). The average age at diagnosis for OvGCT is 50 years: 12% of OvGCT patients are younger than 30 years and 57% are between the ages of 30 and 59 years. OvGCTs are usually slow growing and often do not lead to mortality, so prevalence is high relative to incidence. Mean overall survival (OS) has been shown to be about 13 years (11-15 years) from time at diagnosis with a 10-year OS of 90%.

OvGCTs tend to recur late after primary surgery and primary platinum-based therapy. Therecurrence rate in case series was 32% to 44% (longest reported time to recurrence was 40 years). Median recurrence free survival was 8.4 years (6.8-9.9 years).

OvGCTs originate from early ovarian mesenchyma and are composed of granulosa cells, thecacells, and fibroblasts.

Hyperestrogenism is reported in patients with OvGCT and is related to tumor production of estrogens, anti-Mullerian hormone (AMH), and inhibin A and B. This hormone production differentiates OvGCT from serous ovarian cancer and could serve as a tumor-specific pharmacodynamic marker.

There are currently no Food and Drug Administration (FDA) approved therapies for OvGCT. Surgery, possibly with adjuvant chemotherapy, is the mainstay of initial treatment. Adjuvant chemotherapy is typically with a platinum-based regimen: paclitaxel-carboplatin, cyclophosphamide-cisplatin, or bleomycin-etoposide-cisplatin. Objective Response Rate (ORR) from any monotherapy in the relapse setting is low. Furthermore, combination therapy in the relapse setting has failed to show an improvement in Progression Free Survival (PFS) rates. Therefore, there is an unmet need in this patient population for an agent with a higher response rate and the potential to improve PFS.

### BRIEF SUMMARY OF THE INVENTION

Methods for treating ovarian cancer comprising administering to a patient in need thereof about 50 mg to about 600 mg per day of nirogacestat or a pharmaceutically acceptable salt thereof are provided herein.

Methods for treating ovarian cancer comprising administering to a patient in need thereof a dosage form comprising about 10 mg to about 500 mg of nirogacestat or a pharmaceutically acceptable salt thereof are provided herein.

Methods for treating ovarian cancer comprising administering to a patient in need thereof about 10 mg to about 300 mg twice daily of nirogacestat or a pharmaceutically acceptable salt thereof are provided herein. In some aspects, the nirogacestat or a pharmaceutically acceptable salt thereof is administered about 100 mg per day. In some aspects, the nirogacestat or a pharmaceutically acceptable salt thereof is administered about 200 mg per day. In some aspects, the nirogacestat or a pharmaceutically acceptable salt thereof is administered about 300 mg per day.

In some aspects, about 25 mg of nirogacestat or a pharmaceutically acceptable salt thereof is administered to the patient in need thereof at a single time. In some aspects, about 50 mg of nirogacestat or a pharmaceutically acceptable salt thereof is administered to the patient in need thereof at a single time. In some aspects, about 100 mg of nirogacestat or a pharmaceutically acceptable salt thereof is administered to the patient in need thereof at a single time.

In some aspects, the nirogacestat or a pharmaceutically acceptable salt thereof is administered about 50 mg twice daily. In some aspects, the nirogacestat or a pharmaceutically acceptable salt thereof is administered about 100 mg twice daily. In some aspects, the nirogacestat or a pharmaceutically acceptable salt thereof is administered about 150 mg twice daily.

In some aspects, the ovarian cancer is an ovarian granulosa cell tumor. In some aspects, the patient exhibits hyperestrogenism.

In some aspects, the pharmaceutically acceptable salt form is a hydrobromide salt form. In some aspects, the hydrobromide salt form is a dihydrobromide salt form.

In some aspects, the patient is a human.

In some aspects, the nirogacestat or a pharmaceutically acceptable salt thereof is administered orally. In some aspects, the nirogacestat or a pharmaceutically acceptable salt thereof is administered orally as a solid dosage form. In some aspects, the solid dosage form is a tablet or capsule.

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “nirogacestat” refers to the single enantiomer (S)-2-(((S)-6,8-difluoro-1,2,3,4-tetrahydronaphthalen-2-yl)amino)-N-(1-(2-methyl-1-(neopentylamino) propan-2-yl)-1H-imidazol-4-yl)pentanamide.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

In certain aspects, a subject is successfully “treated” for cancer, e.g., multiple myeloma, according to the methods of the present invention if the patient shows one or more of the following: a reduction in the number of or complete absence of cancer cells; relief of one or more symptoms associated with the specific cancer; reduced morbidity and mortality; improvement in quality of life; increased progression-free survival (“PFS”), disease-free survival (“DFS”), overall survival (“OS”), metastasis-free survival (“MFS”), complete response (“CR”), minimal residual disease (“MRD”), partial response (“PR”), stable disease (“SD”), a decrease in progressive disease (“PD”), an increased time to progression (“TTP”), or any combination thereof. In some aspects, nationally or internationally accepted standards of treatment outcomes in a given cancer can be used to determine whether the therapeutically effective amount nirogacestat or a pharmaceutically acceptable salt thereof meets any of these particular endpoints (e.g., CR, PFS, PR).

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The term “pharmaceutically-acceptable salts” refers to the relatively non-toxic, inorganic and organic acid addition salts of Compound A or Compound B. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66: 1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain aspects, the compounds of the present invention may contain one or more acidic functional groups and, thus, are capable of forming pharmaceutically acceptable salts with pharmaceutically acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

The terms “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

### II. Methods of Treatment

Methods for treating ovarian cancer comprising administering to a patient in need thereof about 50 mg to about 600 mg per day of nirogacestat or a pharmaceutically acceptable salt thereof are provided herein. In some aspects, the nirogacestat or a pharmaceutically acceptable salt thereof is administered about 50 mg to about 575 mg per day, about 75 mg to about 575 mg per day, about 100 mg to about 575 mg per day, about 125 mg to about 575 mg per day, about 150 mg to about 575 mg per day, about 175 mg to about 575 mg per day, about 200 mg to about 575 mg per day, about 225 mg to about 575 mg per day, about 250 mg to about 575 mg per day, about 275 mg to about 575 mg per day, about 300 mg to about 575 mg per day, about 325 mg to about 575 mg per day, about 350 mg to about 575 mg per day, about 375 mg to about 575 mg per day, about 400 mg to about 575 mg per day, about 425 mg to about 575 mg per day, about 450 mg to about 575 mg per day, about 475 mg to about 575 mg per day, about 500 mg to about 575 mg per day, or about 525 mg to about 575 mg per day. In some aspects, the nirogacestat or a pharmaceutically acceptable salt thereof is administered about 50 mg per day, about 75 mg per day, about 100 mg per day, about 125 mg per day, about 150 mg per day, about 175 mg per day, about 200 mg per day, about 225 mg per day, about 250 mg per day, about 275 mg per day, about 300 mg per day, about 325 mg per day, about 350 mg per day, about 375 mg per day, about 400 mg per day, about 425 mg per day, about 450 mg per day, about 475 mg per day, about 500 mg per day, about 525 mg per day, about 550 mg per day, about 575 mg per day, or about 600 mg per day,

Methods for treating ovarian cancer comprising administering to a patient in need thereof a dosage form comprising about 10 mg to about 500 mg of nirogacestat or a pharmaceutically acceptable salt thereof is provided herein. In some aspects, the nirogacestat or a pharmaceutically acceptable salt thereof is administered about 10 mg to about 475 mg at a single time, about 25 mg to about 475 mg at a single time, about 50 mg to about 475 mg at a single time, about 75 mg to about 475 mg at a single time, about 100 mg to about 475 mg at a single time, about 125 mg to about 475 mg at a single time, about 150 mg to about 475 mg at a single time, about 175 mg to about 475 mg at a single time, about 200 mg to about 475 mg at a single time, about 225 mg to about 475 mg at a single time, about 250 mg to about 475 mg at a single time, about 275 mg to about 475 mg at a single time, about 300 mg to about 475 mg at a single time, about 325 mg to about 475 mg at a single time, about 350 mg to about 475 mg at a single time, about 375 mg to about 475 mg at a single time, about 400 mg to about 475 mg at a single time, or about 425 mg to about 475 mg at a single time. In some aspects, the nirogacestat or a pharmaceutically acceptable salt thereof is administered about 10 mg at a single time, about 25 mg at a single time, about 50 mg at a single time, about 75 mg at a single time, about 100 mg at a single time, about 125 mg at a single time, about 150 mg at a single time, about 175 mg at a single time, about 200 mg at a single time, about 225 mg at a single time, about 250 mg at a single time, about 275 mg at a single time, about 300 mg at a single time, about 325 mg at a single time, about 350 mg at a single time, about 375 mg at a single time, about 400 mg at a single time, about 425 mg at a single time, about 450 mg at a single time, about 475 mg at a single time, or about 500 mg at a single time.

Methods for treating ovarian cancer comprising administering to a patient in need thereof about 10 mg to about 300 mg twice daily of nirogacestat or a pharmaceutically acceptable salt thereof are provided herein. In some aspects, the nirogacestat or a pharmaceutically acceptable salt thereof is administered about 10 mg to about 275 mg twice daily, about 25 mg to about 275 mg twice daily, about 50 mg to about 275 mg twice daily, about 75 mg to about 275 mg twice daily, about 100 mg to about 275 mg twice daily, about 125 mg to about 275 mg twice daily, about 150 mg to about 275 mg twice daily, about 175 mg to about 275 mg twice daily, about 200 mg to about 275 mg twice daily, or about 225 mg to about 275 mg twice daily. In some aspects, the nirogacestat or a pharmaceutically acceptable salt thereof is administered about 10 mg twice daily, about 25 mg twice daily, about 50 mg twice daily, about 75 mg twice daily, about 100 mg twice daily, about 125 mg twice daily, about 150 mg twice daily, about 175 mg twice daily, about 200 mg twice daily, about 225 mg twice daily, about 250 mg twice daily, about 275 mg twice daily, or about 300 mg twice daily.

In some aspects, the pharmaceutically acceptable salt form is a hydrobromide salt form. In some aspects, the hydrobromide salt form is a dihydrobromide salt form.

Methods for treating ovarian cancer comprising administering to a patient in need thereof about 50 mg to about 600 mg per day of nirogacestat dihydrobromide are provided herein. In some aspects, the nirogacestat dihydrobromide is administered about 50 mg to about 575 mg per day, about 75 mg to about 575 mg per day, about 100 mg to about 575 mg per day, about 125 mg to about 575 mg per day, about 150 mg to about 575 mg per day, about 175 mg to about 575 mg per day, about 200 mg to about 575 mg per day, about 225 mg to about 575 mg per day, about 250 mg to about 575 mg per day, about 275 mg to about 575 mg per day, about 300 mg to about 575 mg per day, about 325 mg to about 575 mg per day, about 350 mg to about 575 mg per day, about 375 mg to about 575 mg per day, about 400 mg to about 575 mg per day, about 425 mg to about 575 mg per day, about 450 mg to about 575 mg per day, about 475 mg to about 575 mg per day, about 500 mg to about 575 mg per day, or about 525 mg to about 575 mg per day. In some aspects, the nirogacestat dihydrobromide is administered about 50 mg per day, about 75 mg per day, about 100 mg per day, about 125 mg per day, about 150 mg per day, about 175 mg per day, about 200 mg per day, about 225 mg per day, about 250 mg per day, about 275 mg per day, about 300 mg per day, about 325 mg per day, about 350 mg per day, about 375 mg per day, about 400 mg per day, about 425 mg per day, about 450 mg per day, about 475 mg per day, about 500 mg per day, about 525 mg per day, about 550 mg per day, about 575 mg per day, or about 600 mg per day,

Methods for treating ovarian cancer comprising administering to a patient in need thereof a dosage form comprising about 10 mg to about 500 mg of nirogacestat dihydrobromide is provided herein. In some aspects, the nirogacestat dihydrobromide is administered about 10 mg to about 475 mg at a single time, about 25 mg to about 475 mg at a single time, about 50 mg to about 475 mg at a single time, about 75 mg to about 475 mg at a single time, about 100 mg to about 475 mg at a single time, about 125 mg to about 475 mg at a single time, about 150 mg to about 475 mg at a single time, about 175 mg to about 475 mg at a single time, about 200 mg to about 475 mg at a single time, about 225 mg to about 475 mg at a single time, about 250 mg to about 475 mg at a single time, about 275 mg to about 475 mg at a single time, about 300 mg to about 475 mg at a single time, about 325 mg to about 475 mg at a single time, about 350 mg to about 475 mg at a single time, about 375 mg to about 475 mg at a single time, about 400 mg to about 475 mg at a single time, or about 425 mg to about 475 mg at a single time. In some aspects, the nirogacestat dihydrobromide is administered about 10 mg at a single time, about 25 mg at a single time, about 50 mg at a single time, about 75 mg at a single time, about 100 mg at a single time, about 125 mg at a single time, about 150 mg at a single time, about 175 mg at a single time, about 200 mg at a single time, about 225 mg at a single time, about 250 mg at a single time, about 275 mg at a single time, about 300 mg at a single time, about 325 mg at a single time, about 350 mg at a single time, about 375 mg at a single time, about 400 mg at a single time, about 425 mg at a single time, about 450 mg at a single time, about 475 mg at a single time, or about 500 mg at a single time.

Methods for treating ovarian cancer comprising administering to a patient in need thereof about 10 mg to about 300 mg twice daily of nirogacestat dihydrobromide are provided herein. In some aspects, the nirogacestat dihydrobromide is administered about 10 mg to about 275 mg twice daily, about 25 mg to about 275 mg twice daily, about 50 mg to about 275 mg twice daily, about 75 mg to about 275 mg twice daily, about 100 mg to about 275 mg twice daily, about 125 mg to about 275 mg twice daily, about 150 mg to about 275 mg twice daily, about 175 mg to about 275 mg twice daily, about 200 mg to about 275 mg twice daily, or about 225 mg to about 275 mg twice daily. In some aspects, the nirogacestat or dihydrobromide is administered about 10 mg twice daily, about 25 mg twice daily, about 50 mg twice daily, about 75 mg twice daily, about 100 mg twice daily, about 125 mg twice daily, about 150 mg twice daily, about 175 mg twice daily, about 200 mg twice daily, about 225 mg twice daily, about 250 mg twice daily, about 275 mg twice daily, or about 300 mg twice daily.

In some aspects, the nirogacestat or pharmaceutically acceptable salt thereof (e.g., a dihydrobromide salt form) is administered one, two, three, or four times per day. In some aspects, the nirogacestat or pharmaceutically acceptable salt thereof (e.g., a dihydrobromide salt form) is administered twice daily. If the nirogacestat or pharmaceutically acceptable salt thereof (e.g., a dihydrobromide salt form) is administered more than one time daily, the dose administered each time can be the same or different. For example, if a total daily dose of about 300 mg nirogacestat or pharmaceutically acceptable salt thereof (e.g., a dihydrobromide salt form) is administered twice daily, the patient could receive either two 150 mg doses (e.g., one 150 mg dose at 8 am and one 150 mg dose at 8 pm) or a 100 mg dose in the morning and a 200 mg dose in the evening. Each dose can also consist of more than one solid dosage form. For example, a 150 mg individual dose (i.e., the morning dose of a 300 mg total daily dose to be administered as two separate doses) could be administered as three 50 mg tablets.

In some aspects, the ovarian cancer is an ovarian granulosa cell tumor. In some aspects, the patient exhibits hyperestrogenism.

In some aspects, the patient is a human. In some aspects, the nirogacestat or a pharmaceutically acceptable salt thereof (e.g., a dihydrobromide salt form) is administered orally. In some aspects, the nirogacestat or a pharmaceutically acceptable salt thereof (e.g., a dihydrobromide salt form) is administered orally as a solid dosage form. In some aspects, the solid dosage form is a tablet or capsule. In some aspects, the solid dosage form is a tablet.

### EXAMPLES

### Example 1: Phase 2 Trial of Nirogacestat in Adult Patients With Relapsed/Refractory Ovarian Granulosa Cell Tumor (OvGCT)

Overall Design: A multi-center, single-arm, Phase 2 open label treatment study will be conducted to determine the efficacy, safety, tolerability, and pharmacokinetics of nirogacestat in adult participants with relapsed/refractory OvGCT. Participants must have received at least one prior course of systemic therapy for OvGCT and have measurable disease by RECIST v1.1 to meet the eligibility criteria.

Participants will be screened up to 28 days prior to the first dose of study treatment (nirogacestat) and full eligibility will be based on the inclusion and exclusion criteria.

Eligible participants will be enrolled in the study using the Interactive Response Technology (IRT) system following all pre-dose baseline assessments at Cycle 1 Day 1 (C1D1). Following C1D1, participants will return to the clinic for scheduled study visits monthly for the first year and then every 3 months thereafter until end of study participation.

Participants who discontinue study treatment early for any reason should return to the clinic for an End of Treatment (EOT) visit within 7 days after the Investigator determines study treatment will no longer be used and then again for a safety Follow-up (FUP) visit 30 days (+7 days) after the last dose of study treatment.

Based on current enrollment assumptions, the estimated study duration is 5 years, and all participants will be followed for at least 2 years (unless the study is prematurely stopped due to futility).

Number of Participants: Approximately 43 participants.

Treatment groups and duration: At C1D1 participants will be enrolled in the study using the IRT system and will receive 150 mg (orally) twice daily (BID) of study treatment (nirogacestat; 3 × 50 mg tablets), continuously in 28-day cycles.

Participants will remain on study treatment until death, disease progression, discontinuation of study treatment for any reason, the study is stopped by the Sponsor for any reason, or participant qualifies for Sponsor’s Expanded Access.

The Objectives and Endpoints are provided in Table 1.

TABLE 1

Objectives and Endpoints

| Primary Objectives: | Primary Endpoints: |
|---|---|
| To determine the anti-tumor activity of nirogacestat in adult participants with relapsed/refractory OvGCT | Objective response rate, defined as the proportion of participants with Complete Response (CR) + Partial Response (PR) using Response Evaluation Criteria in Solid Tumors (RECIST) v1.1 |
| Secondary Objectives: | Secondary Endpoints: |
| To determine if nirogacestat delays progression or death in OvGCT | Estimate of proportion of participants who have not progressed or died at 6 months follow-up: PFS-6. Progression is defined by RECIST v1.1 |
| To describe overall survival in participants treated with nirogacestat | Estimate of 2-year overall survival, defined as the proportion of participants who have not died after 2 years of follow-up after their firstdose of nirogacestat |
| To determine the effect of nirogacestat on ovarian cancer symptoms measured by Functional Assessment of Cancer Therapy - Ovarian Symptom Index (FOSI) | Change from baseline in FOSI |
| To determine the duration of response | Duration of response rate, defined as the time from response (CR + PR using RECIST v1.1) to disease progression and/or death |
| To determine the pharmacokinetics (PK) of nirogacestat | Serum concentrations of nirogacestat will be measured to evaluate system exposures (Cₘₐₓ, C_{trough} and other PK parameters as data allow) |
| Safety Objectives | Safety Endpoints |
| To characterize the safety and tolerability of nirogacestat at a dose of 150 mg BID in adult participants with relapsed/refractory OvGCT | Key safety endpoints will include incidence of treatment-emergent Adverse Events (TEAEs), changes in clinical laboratory parameters, vital signs, physical examination findings, and electrocardiograms (ECGs) |
| | Tolerability will be assessed according to toxicities graded by National Cancer Institute Common Terminology Criteria for Adverse Events (CTCAE) v5.0 |
| Exploratory Objectives | Exploratory Endpoints |
| To detect FOXL2 C 134W mutation as well as other genomic alterations and correlate these with response | Evaluate Next Generation Sequencing (NGS) status in baseline tumor tissue |
| To detect NICD and candidate biomarkers of response, and to correlate nirogacestat exposure with response | Evaluate change from Baseline: |
| | • Inhibin A&B, Follicle-stimulating hormone (FSH), estradiol, CA-125, and Mullerian Inhibiting Substance (MIS) / AMH |
| | • Circulating tumor DNA (ctDNA) Baseline NICD expression in tumor tissue |

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

In addition to the various embodiments described herein, the present disclosure includes the following embodiments numbered E1 through E22. This list of embodiments is presented as an exemplary list and the application is not limited to these embodiments.

E1. A method for treating ovarian cancer comprising administering to a patient in need thereof about 50 mg to about 600 mg per day of nirogacestat or a pharmaceutically acceptable salt thereof.

E2. A method for treating ovarian cancer comprising administering to a patient in need thereof a dosage form comprising about 10 mg to about 500 mg of nirogacestat or a pharmaceutically acceptable salt thereof.

E3. A method for treating ovarian cancer comprising administering to a patient in need thereof about 10 mg to about 300 mg twice daily of nirogacestat or a pharmaceutically acceptable salt thereof.

E4. The method of any one of E1-E3, wherein the nirogacestat or a pharmaceutically acceptable salt thereof is administered about 100 mg per day.

E5. The method of any one of E1-E3, wherein the nirogacestat or a pharmaceutically acceptable salt thereof is administered about 200 mg per day.

E6. The method of any one of E1-E3, wherein the nirogacestat or a pharmaceutically acceptable salt thereof is administered about 300 mg per day.

E7. The method of any one of E1-E6, wherein about 25 mg of nirogacestat or a pharmaceutically acceptable salt thereof is administered to the patient in need thereof at a single time.

E8. The method of any one of E1-E6, wherein about 50 mg of nirogacestat or a pharmaceutically acceptable salt thereof is administered to the patient in need thereof at a single time.

E9. The method of any one of E1-E6, wherein about 100 mg of nirogacestat or a pharmaceutically acceptable salt thereof is administered to the patient in need thereof at a single time.

E10. The method of any one of E1-E9, wherein the nirogacestat or a pharmaceutically acceptable salt thereof is administered about 50 mg twice daily.

E11. The method of any one of E1-E9, wherein the nirogacestat or a pharmaceutically acceptable salt thereof is administered about 100 mg twice daily.

E12. The method of any one of E1-E9, wherein the nirogacestat or a pharmaceutically acceptable salt thereof is administered about 150 mg twice daily.

E13. The method of any one of E1-E2 and E4-E9, wherein the nirogacestat or pharmaceutically acceptable salt thereof is administered one, two, three, or four times per day.

E14. The method of E 13, wherein the nirogacestat or pharmaceutically acceptable salt thereof is administered twice daily.

E15. The method of any one of E1-E14, wherein the ovarian cancer is an ovarian granulosa cell tumor.

E16. The method of any one of E1-E15, wherein the patient exhibits hyperestrogenism.

E17. The method of any one of E1-E16, wherein the pharmaceutically acceptable salt form is a hydrobromide salt form.

E18. The method of E 17, wherein the hydrobromide salt form is a dihydrobromide salt form.

E19. The method of any one of E1-E18, wherein the patient is a human.

E20. The method of any one of E1-E19, wherein the nirogacestat or a pharmaceutically acceptable salt thereof is administered orally.

E21. The method of E 20, wherein the nirogacestat or a pharmaceutically acceptable salt thereof is administered orally as a solid dosage form.

E22. The method of E21, wherein the solid dosage form is a tablet or capsule.

1. A method for treating ovarian cancer comprising administering to a patient in need thereof about 50 mg to about 600 mg per day of nirogacestat or a pharmaceutically acceptable salt thereof.

2. A method for treating ovarian cancer comprising administering to a patient in need thereof a dosage form comprising about 10 mg to about 500 mg of nirogacestat or a pharmaceutically acceptable salt thereof.

3. A method for treating ovarian cancer comprising administering to a patient in need thereof about 10 mg to about 300 mg twice daily of nirogacestat or a pharmaceutically acceptable salt thereof.

4. The method of claim 1, wherein the nirogacestat or a pharmaceutically acceptable salt thereof is administered about 100 mg per day.

5. The method of claim 1, wherein the nirogacestat or a pharmaceutically acceptable salt thereof is administered about 200 mg per day.

6. The method of claim 1, wherein the nirogacestat or a pharmaceutically acceptable salt thereof is administered about 300 mg per day.

7. The method of claim 1, wherein about 25 mg of nirogacestat or a pharmaceutically acceptable salt thereof is administered to the patient in need thereof at a single time.

8. The method of claim 1, wherein about 50 mg of nirogacestat or a pharmaceutically acceptable salt thereof is administered to the patient in need thereof at a single time.

9. The method of claim 1, wherein about 100 mg of nirogacestat or a pharmaceutically acceptable salt thereof is administered to the patient in need thereof at a single time.

10. The method of claim 1, wherein the nirogacestat or a pharmaceutically acceptable salt thereof is administered about 50 mg twice daily.

11. The method of claim 1, wherein the nirogacestat or a pharmaceutically acceptable salt thereof is administered about 100 mg twice daily.

12. The method of claim 1, wherein the nirogacestat or a pharmaceutically acceptable salt thereof is administered about 150 mg twice daily.

13. The method of claim 1, wherein the nirogacestat or pharmaceutically acceptable salt thereof is administered one, two, three, or four times per day.

14. The method of claim 13, wherein the nirogacestat or pharmaceutically acceptable salt thereof is administered twice daily.

15. The method of claim 1, wherein the ovarian cancer is an ovarian granulosa cell tumor.

16. The method of claim 1, wherein the patient exhibits hyperestrogenism.

17. The method of claim 1, wherein the pharmaceutically acceptable salt form is a hydrobromide salt form.

18. The method of claim 17, wherein the hydrobromide salt form is a dihydrobromide salt form.

19. The method of claim 1, wherein the patient is a human.

20. The method of claim 1, wherein the nirogacestat or a pharmaceutically acceptable salt thereof is administered orally.

21. The method of claim 20, wherein the nirogacestat or a pharmaceutically acceptable salt thereof is administered orally as a solid dosage form.

22. The method of claim 21, wherein the solid dosage form is a tablet or capsule.
